(19) United States
(12) Patent Application Publication
(10) Pub. No.: US 2024/0398354 A1
(43) Pub. Date: Dec. 5, 2024

(54) Wearable Wellness Band

(71) Applicant: Daryn Amari Fields, Albany, NY (US)

(72) Inventor: Daryn Amari Fields, Albany, NY (US)

(21) Appl. No.: 18/327,901

(22) Filed: Jun. 2, 2023

(51) Int. Cl. A61B 5/00; A61B 5/0205

(52) U.S. Cl. CPC A61B 5/7275; A61B 5/02055; A61B 5/4806; A61B 5/6802; A61B 5/74; A61B 5/7282

(57) ABSTRACT

The present invention discloses a wearable wellness band that tracks health data and detects early signs of illness or sickness in humans. The invention is a wearable band equipped with various sensors, including those for heart rate, blood pressure, temperature, GSR, oxygen saturation, step count, EMG, altitude, air quality, UV exposure, calories burn, stress level, humidity, and sleep patterns. It utilizes advanced algorithms and data analytics techniques to process and analyze the collected data. By comparing the data against established health baselines and patterns, the band can identify deviations, anomalies, or early signs of illness. It provides timely notifications and recommendations to the user, promoting early intervention and proactive health management.

### TECHNICAL FIELD

The present invention relates to the wearable medical technology, and in particular a wearable wellness band that tracks health data and detects early signs of illness or sickness in humans.

### BACKGROUND ART

Medical technology is helping patients and healthcare professionals in new and innovative ways. It is now known that technology has transformed people's lives. Whether its smartphones or smart cars and houses, tech has crept its way into nearly every facet of our day to day, including our healthcare. And while our minds may leap to thoughts of AI and robotic surgery, some medical technology is a whole lot smaller. From the runner checking her heartrate at the end of a 5 k to the guy administering a self-EKG after a series of heart palpations, all from a smartwatch, wearable tech is making a big difference.

The rise of wearable tech in healthcare is changing the way we're able to monitor our own health. Now, sensors can alert a diabetic patient when their blood sugar is getting low, and smart contact lenses can give the vision-impaired the opportunity to see. While these technologies may be small, their impact can be huge. Continue reading to discover the top wearable technologies in healthcare and learn which careers are at the forefront of the medical technology revolution.

The emergence of wearable tech in healthcare offers solutions to diverse ailments, including vision loss, hearing loss, and physical disabilities. From the science-fiction feel of exoskeleton technology to the ubiquity of smartwatches, the field is expansive. Below are some of the most used wearable/smart devices being used in healthcare today.

The U.S. Pat. No. 9,775,520B2 dated 2015 Nov. 3 discloses a wearable personal monitoring system. A system includes one or more sensors to detect activities of a mobile object; and a processor coupled to the sensor and the wireless transceiver to classify sequences of motions into groups of similar postures each represented by a model and to apply the models to identify an activity of the object.

The U.S. Pat. No. 9,107,586B2 of 2014 May 16 discloses a fitness monitoring. A mobile system for a user includes a telephone having one or more sensors to capture fitness data or vital sign data, the telephone having a wireless transceiver coupled to the processor to communicate fitness or vital sign data over a personal area network; and a processor coupled to the personal area network to process the fitness or vital sign data.

However, the above prior art documents do not describe a wearable wellness band that tracks health data and detects early signs of illness or sickness in humans as presented in the invention.

### Objects of the Invention

The main object of the present invention is to develop a wearable wellness band that tracks health data and detects early signs of illness or sickness in humans.

The other object is to provide users with real-time monitoring of vital signs and health parameters.

The other object is to detect and alert users to early signs of illness or abnormal health conditions.

The other object is to enable proactive health management and preventive care.

The other object is to improve overall well-being and quality of life.

The foregoing and other objects, features, and advantages of the invention will be apparent from the following, more particular description of the preferred embodiments of the invention, as illustrated in the accompanying drawings.

### DESCRIPTION OF THE INVENTION

The following description is presented to enable any person skilled in the art to make and use the invention as claimed and is provided in the context of the particular examples discussed below, variations of which will be readily apparent to those skilled in the art. In the interest of clarity, not all features of an actual implementation are described in this specification. It will be appreciated that in the development of any such actual implementation (as in any development project), design decisions must be made to achieve the designers' specific goals (e.g., compliance with system- and business-related constraints), and that these goals will vary from one implementation to another.

The terminology used herein is for the purpose of describing particular example embodiments only and is not intended to be limiting. As used herein, the singular forms “a”, “an” and “the” may be intended to include the plural forms as well, unless the context clearly indicates otherwise. In other words, an element or step recited in the singular and preceded by the word “a” or “an” should be understood as not necessarily excluding the plural of the elements or steps. Further, references to “one embodiment” or “one implementation” are not intended to be interpreted as excluding the existence of additional embodiments or implementations that also incorporate the recited features.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps. In other words, unless explicitly stated to the contrary, embodiments “comprising” or “having” an element or a plurality of elements having a particular property can include additional elements not having that property. In other words, the terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. In other words, the use of “including,” “comprising,” “having,” “containing,” “involving,” and variations thereof, is meant to encompass the items listed thereafter and additional items.

Further, references to “one embodiment” or “one implementation” are not intended to be interpreted as excluding the existence of additional embodiments or implementations that also incorporate the recited features. The term “exemplary” is intended to mean “an example of”.

When introducing elements of aspects of the disclosure or the examples thereof, the articles “a,” “an,” “the,” and “said” are intended to mean that there are one or more of the elements. In other words, the indefinite articles “a”, “an”, “the”, and “said” as used in the specification and in the claims, unless clearly indicated to the contrary, should be understood to mean “at least one.” The use of the expression “at least” or “at least one” suggests the use of one or more elements or ingredients or quantities, as the use may be in the embodiment of the disclosure to achieve one or more of the desired objects or results.

To achieve the above-mentioned object, is there a requirement to develop a wearable wellness band that tracks health data and detects early signs of illness or sickness in humans.

The invention addresses the challenge of early detection and monitoring of health conditions. By continuously tracking vital signs and analyzing the data, it aims to identify potential health issues at an early stage, allowing for timely intervention and preventive measures.

The present invention discloses a wearable wellness band that tracks health data and detects early signs of illness or sickness in humans.

The invention describes a wearable band product that tracks health data and detects early signs of illness or sickness. It combines sensor technology, data analytics, AI technology and wearable technology to provide users with valuable insights into their health status.

The invention is a wearable band equipped with various sensors, including those for heart rate, blood pressure, temperature, GSR, oxygen saturation, step count, EMG, altitude, air quality, UV exposure, calories burn, stress level, humidity, and sleep patterns. It utilizes advanced algorithms and data analytics techniques to process and analyze the collected data.

By comparing the data against established health baselines and patterns, the band can identify deviations, anomalies, or early signs of illness. It provides timely notifications and recommendations to the user, promoting early intervention and proactive health management.

The wearable wellness band that provides a continuous health monitoring through wearable technology; integrates multiple sensors to track vital signs, including heart rate, blood pressure, temperature, and sleep patterns; comprises an advanced algorithms and data analytics for real-time analysis of health data; provides an early detection and notification of potential health issues or abnormalities and comprises personalized recommendations and proactive health management based on individual health data.

### ADVANTAGES IN THE INDUSTRY

1. Early Detection: The product enables early detection of health issues, allowing for timely intervention and improved health outcomes.
2. Personalized Health Monitoring: The wearable band provides personalized health data and insights, empowering individuals to monitor and manage their well-being effectively.
3. Convenience and Accessibility: The wearable nature of the product ensures continuous monitoring without disruption to daily activities, making it convenient and easily accessible for users.
4. Data-Driven Insights: The integration of data analytics enables the identification of patterns and trends, providing valuable insights for individuals and healthcare professionals.

While certain novel features of the present invention have been shown and described, it will be understood that various omissions, substitutions, and changes in the forms and details of the device illustrated and in its operation can be made by those skilled in the art without departing from the spirit of the invention.

### INDUSTRIAL APPLICATION

The present invention applies to the wearable medical technology. It brings on-board a wearable wellness band that tracks health data and detects early signs of illness or sickness in humans. The product enables early detection of health issues, allowing for timely intervention and improved health outcomes, personalized health data and insights, empowering individuals to monitor and manage their well-being effectively. The wearable nature of the product ensures continuous monitoring without disruption to daily activities, making it convenient and easily accessible for users. The integration of data analytics enables the identification of patterns and trends, providing valuable insights for individuals and healthcare professionals.

What is claimed:

1. A wearable wellness band that tracks health data and detects early signs of illness or sickness in humans, comprising:
i. A sensor technology,
ii. data analytics, and
iii. an AI technology;
wherein the said wearable technology is designed to provide users with valuable insights into their health status.

2. The wearable wellness band according to claim 1, wherein the wearable band is equipped with various sensors suited for those for heart rate, blood pressure, temperature, GSR, oxygen saturation, step count, EMG, altitude, air quality, UV exposure, calories burn, stress level, humidity, and sleep patterns.

3. The wearable wellness band according to claim 1, wherein the said device is designed to utilize advanced algorithms and data analytics techniques to process and analyze the collected data.

4. The wearable wellness band according to claim 3, wherein the said device is utilized by comparing the data against established health baselines and patterns, the band can identify deviations, anomalies, or early signs of illness.

5. The wearable wellness band according to claim 4, wherein the said device provides timely notifications and recommendations to the user, promoting early intervention and proactive health management.

6. The wearable wellness band according to claim 5, wherein the said device provides:
i. provides a continuous health monitoring through wearable technology;
ii. integrates multiple sensors to track vital signs, including heart rate, blood pressure, temperature, and sleep patterns;
iii. comprises an advanced algorithms and data analytics for real-time analysis of health data;
iv. provides an early detection and notification of potential health issues or abnormalities;
v. comprises personalized recommendations and proactive health management based on individual health data.
